(19) United States
(12) Patent Application Publication
(10) Pub. No.: US 2018/0164243 A1
(43) Pub. Date: Jun. 14, 2018

(54) BIOSENSOR CHIP AND BIOSENSOR DEVICE

(71) Applicant: NITTO DENKO CORPORATION, Ibaraki-shi, Osaka (JP)

(72) Inventors: Shunsuke NOUMI, Osaka (JP); Kenichi YAMAMOTO, Osaka (JP); Yuichi ABE, Osaka (JP); Kazuaki MOCHIDA, Osaka (JP)

(73) Assignee: NITTO DENKO CORPORATION, Ibaraki-shi, Osaka (JP)

(21) Appl. No.: 15/577,807

(22) PCT Filed: Jun. 6, 2016
(86) PCT No.: PCT/JP2016/002723

§ 371 Date: Nov. 29, 2017

(30) Foreign Application Priority Data
Jun. 5, 2015 (JP) 2015-115068

(51) Int. Cl. G01N 27/327; A61B 5/145

(52) U.S. Cl. CPC G01N 27/327; A61B 5/14532

(57) ABSTRACT

A biosensor chip (1) includes: an electrode substrate (11) having a first principal surface (11a) provided with an electrode (151, 152); a cover film (14) opposed to the first principal surface (11a); a spacer layer (13) disposed between the electrode substrate (11) and the cover film (14), the spacer layer having a slit (13a) provided in a region positionally corresponding at least to the electrode (151, 152), the spacer layer serving as a bonding member to join the substrate (11) and the cover film (14) together; and a hydrophilic filter (12) disposed between the spacer layer (13) and the substrate (11) and covering at least a portion of the electrode (151, 152), the portion of the electrode positionally corresponding to the slit (13a). A zone defined by the cover film (14), the slit (13a) of the spacer layer (13), and the electrode substrate (11) serves as a sample channel.

### TECHNICAL FIELD

The present invention relates to biosensor chips and biosensor devices and relates to, for example, a biosensor chip and a biosensor device that are used to measure the concentration of a component in a blood sample.

### BACKGROUND ART

The number of diabetes patients has increased in recent years. The basic approach for treatment of diabetes is to control the blood-glucose level, and insulin is typically used for control of the blood-glucose level. Whether insulin needs to be administered into a diabetes patient is determined on the basis of the blood-glucose level of the patient. Various devices for self monitoring of blood glucose (SMBG) have thus been proposed to allow diabetes patients to easily check their blood-glucose level in their daily life.

A commonly-used device for SMBG is a biosensor device whose operating principle is based on an electrochemical method. Such a biosensor device for SMBG is used, for example, with a disposable biosensor chip attached to the device body. The operating principle of the device is as follows. When blood is applied dropwise or introduced to an electrode portion of the biosensor chip, an enzyme provided beforehand in the biosensor chip oxidizes blood sugar (glucose), and the enzyme itself is reduced. The enzyme in a reduced state undergoes oxidation-reduction reaction with an electron carrier (oxidized state) provided beforehand in the biosensor chip and thereby brings the electron carrier into a reduced state. The electron carrier in a reduced state reaches an electrode surface on which a potential is imposed, and the electron carrier undergoes oxidation reaction at the electrode surface, generating a current flowing between the electrodes. The flowing current depends on the glucose concentration in the blood. The glucose concentration in the blood (blood-glucose level) can thus be indirectly measured by the current value.

As described above, the blood-glucose level measurement necessitates bringing a blood sample into contact with an electrode of a biosensor chip. However, when red blood cells in the blood sample adhere to the electrode, that portion of the electrode surface to which the red blood cells have adhered is insulated, and the effective area of the electrode is thus reduced. This results in a decrease in the current value to be detected, causing an error in the blood-glucose level measurement.

Under the above circumstances, biosensor devices capable of reducing the error as described above have been proposed (Patent Literatures 1 and 2). These devices are configured to determine the hematocrit level (the proportion of the volume of red blood cells in blood) of a blood sample from the flowability of the blood and correct a measurement result of the blood-glucose level on the basis of the determined hematocrit level (hematocrit correction).

### CITATION LIST

### Patent Literature

Patent Literature 1: JP 2006-215034 A
Patent Literature 2: JP 2011-145291 A

### SUMMARY OF INVENTION

### Technical Problem

However, the hematocrit correction has been pointed out to entail the risk of overcorrection and is still insufficient to improve the measurement accuracy. For example, there is a risk that the patient will improperly administer insulin on the basis of an inaccurate measurement result deviating from the true blood-glucose level. It cannot be denied that such improper administration can lead to a serious medical accident which adversely affects the body of the patient. The improvement in the accuracy of the blood-glucose level measurement can thus be considered an important medical issue in terms of treatment of diabetes which is accompanied by various complications such as brain infarction, cardiac infarction, and neurological disorder.

It is therefore an object of the present invention to provide a biosensor chip and a biosensor device with which the concentration of a component (such as blood glucose) in a sample to be sensed such as a blood sample can be measured with improved accuracy.

### Solution to Problem

A biosensor chip according to a first aspect of the present invention includes:

a substrate having a first principal surface provided with an electrode;

a cover film opposed to the first principal surface of the substrate; and

a spacer layer disposed between the substrate and the cover film and serving as a bonding member to join the substrate and the cover film together, wherein

the spacer layer is provided with a slit forming: a sample inlet orifice provided at a peripheral surface of a laminate of the substrate, the spacer layer, and the cover film; and a sample channel for delivering a sample to the electrode by capillary action, and

a hydrophilic filter is provided between the slit of the spacer layer and a sample sensing portion of the electrode of the substrate.

A biosensor chip according to a second aspect of the present invention includes:

a substrate having a first principal surface provided with an electrode;

a cover film opposed to the first principal surface of the substrate;

a spacer layer disposed between the substrate and the cover film, the spacer layer having a slit provided in a region positionally corresponding at least to the electrode, the spacer layer serving as a bonding member to join the substrate and the cover film together; and

a hydrophilic filter disposed between the spacer layer and the substrate and covering at least a portion of the electrode, the portion of the electrode positionally corresponding to the slit, wherein

a zone defined by the cover film, the slit of the spacer layer, and the substrate serves as a sample channel.

A biosensor chip according to a third aspect of the present invention includes:

a substrate having a first principal surface provided with a sensing portion that senses a blood sample;

a cover film opposed to the first principal surface of the substrate;

a spacer layer disposed between the substrate and the cover film, the spacer layer having a sample channel into which the blood sample is introduced by capillary action, the spacer layer serving as a bonding member to join the substrate and the cover film together; and

a hydrophilic filter disposed between the spacer layer and the substrate and located at a position through which the blood sample passes to reach the sensing portion.

The present invention also provides a biosensor device including:

a device body; and

the above biosensor chip according to the present invention, the biosensor chip being detachably attached to the device body, wherein

the device body includes:

a detection portion that detects a substance in a sample on the basis of a value of a current flowing between a pair of electrodes of the biosensor chip;

an analysis portion that analyzes a detection result obtained by the detection portion; and

a display portion that displays as a measurement value an analysis result obtained by the analysis portion.

### Advantageous Effects of Invention

When a sample to be sensed by the biosensor chip according to present invention is a blood sample, the blood sample moving in the sample channel toward the electrode or sensing portion passes through the hydrophilic filter, and thus penetration of blood components such as red blood cells to the electrode or sensing portion can be prevented. The value determined from a current flowing in the electrode or the sensing result obtained by the sensing portion is therefore an accurate value or result which is less affected, for example, by red blood cells. Thus, the use of the biosensor chip according to the present invention makes it possible, for example, to measure the concentration of a component (blood glucose, for example) in a blood sample with improved accuracy.

The biosensor device according to the present invention includes the biosensor chip according to the present invention which provides the above effect and is thus capable, for example, of measuring the concentration of a component (blood glucose, for example) in a blood sample with improved accuracy.

### BRIEF DESCRIPTION OF DRAWINGS

FIG. 1A is a schematic exploded perspective view showing a configuration example of a biosensor chip according to an embodiment of the present invention.

FIG. 1B is a cross-sectional view along the line I-I of FIG. 1A.

FIG. 2A is a schematic exploded perspective view showing another configuration example of the biosensor chip according to the embodiment of the present invention.

FIG. 2B is a cross-sectional view along the line II-II of FIG. 2A.

FIG. 3A is a schematic exploded perspective view showing still another configuration example of the biosensor chip according to the embodiment of the present invention.

FIG. 3B is a cross-sectional view along the line III-III of FIG. 3A.

FIG. 4A is a schematic exploded perspective view showing still another configuration example of the biosensor chip according to the embodiment of the present invention.

FIG. 4B is a cross-sectional view along the line IV-IV of FIG. 4A.

FIG. 5A is a schematic exploded perspective view showing still another configuration example of the biosensor chip according to the embodiment of the present invention.

FIG. 5B is a cross-sectional view along the line V-V of FIG. 5A.

FIG. 6 is a schematic view of a biosensor device according to an embodiment of the present invention.

FIG. 7 is a cross-sectional view of a test cell used in Reference Example A.

FIG. 8 is a top view of the test cell used in Reference Example A.

FIG. 9 is a cross-sectional view showing a state where a filter is placed in the test cell used in Reference Example A.

FIG. 10 is a top view showing a state where a filter is placed in a test cell used in Reference Example B.

FIG. 11A is a cross-sectional view along the line A-A of FIG. 10.

FIG. 11B is a cross-sectional view along the line B-B of FIG. 10.

FIG. 12 is a top view showing a state where a filter is placed in another test cell used in Reference Example B.

### DESCRIPTION OF EMBODIMENTS

Hereinafter, embodiments of the present invention will be described. The following description is not intended to limit the present invention.

A biosensor chip according to an embodiment of the present invention includes: a substrate having a first principal surface provided with an electrode; a cover film opposed to the first principal surface of the substrate; a spacer layer disposed between the substrate and the cover film, the spacer layer having a slit provided in a region positionally corresponding at least to the electrode, the spacer layer serving as a bonding member to join the substrate and the cover film together; and a hydrophilic filter disposed between the spacer layer and the substrate and covering at least a portion of the electrode, the portion of the electrode positionally corresponding to the slit. A zone defined by the cover film, the slit of the spacer layer, and the substrate serves as a sample channel. In the biosensor chip according to the present embodiment, the position where the sample inlet orifice of the sample channel is provided is not limited. The following will describe an example where the sample inlet orifice is that opening of the sample channel which lies at the peripheral surface of a laminate of the substrate, the spacer layer, and the cover film.

The present embodiment will be described with an example where the sample to be sensed is a blood sample.

The biosensor chip according to the present embodiment has a configuration in which the opening of the sample channel at the peripheral surface of the laminate of the substrate, the spacer layer, and the cover film serves as a sample inlet orifice leading to the sample channel and in which the sample is introduced into the sample channel by so-called capillary action. In the biosensor chip according to the present embodiment which has such a configuration, a blood sample moving in the sample channel from the sample inlet orifice to the electrode passes through the hydrophilic filter, and thus penetration of red blood cells to the electrode can be prevented. The value determined from a current flowing in the electrode is therefore an accurate value less affected by red blood cells. The use of the biosensor chip according to the present embodiment thus makes it possible to measure the concentration of a specific component (blood glucose, for example) in a blood sample with improved accuracy. Conventional biosensor chips configured to introduce a blood sample into a sample channel by capillary action require hydrophilization of a member defining the wall surface of the sample channel, such as hydrophilization of a sample channel-facing portion of the cover film, for the purpose of promoting the capillary action. By contrast, the biosensor chip according to the present embodiment does not require hydrophilization of a member defining the wall surface of the sample channel because the filter, which is provided in the sample channel to lie over the electrode reached by the blood sample and cover at least that portion of the electrode which positionally corresponds to the slit, is hydrophilic. Additionally, the biosensor chip according to the present embodiment has the advantage of being capable of more efficiently delivering the blood sample to the electrode than conventional biosensor chips in which a member defining the wall surface of the sample channel is hydrophilized. When it is stated herein that a spacer layer has a slit provided in a region positionally corresponding to an electrode, this is intended to refer to, for example, a configuration in which the slit is provided in the spacer layer in such a manner that the slit overlaps at least a portion of the electrode when a laminate of the substrate and the spacer layer is viewed in the lamination direction. That portion of the electrode which positionally corresponds to the slit is, for example, a portion of the electrode that overlaps the slit when a laminate of the substrate and the spacer layer is viewed in the lamination direction. When it is stated that a hydrophilic filter covers at least that portion of the electrode which positionally corresponds to the slit, this is intended to encompass both a configuration in which the hydrophilic filter covers directly (is in contact with) the portion of the electrode and a configuration in which the hydrophilic filter covers indirectly (is not in contact with) the portion of the electrode.

Hereinafter, examples of the configuration of the biosensor chip according to the present embodiment will be described with reference to the drawings.

[First Configuration Example]

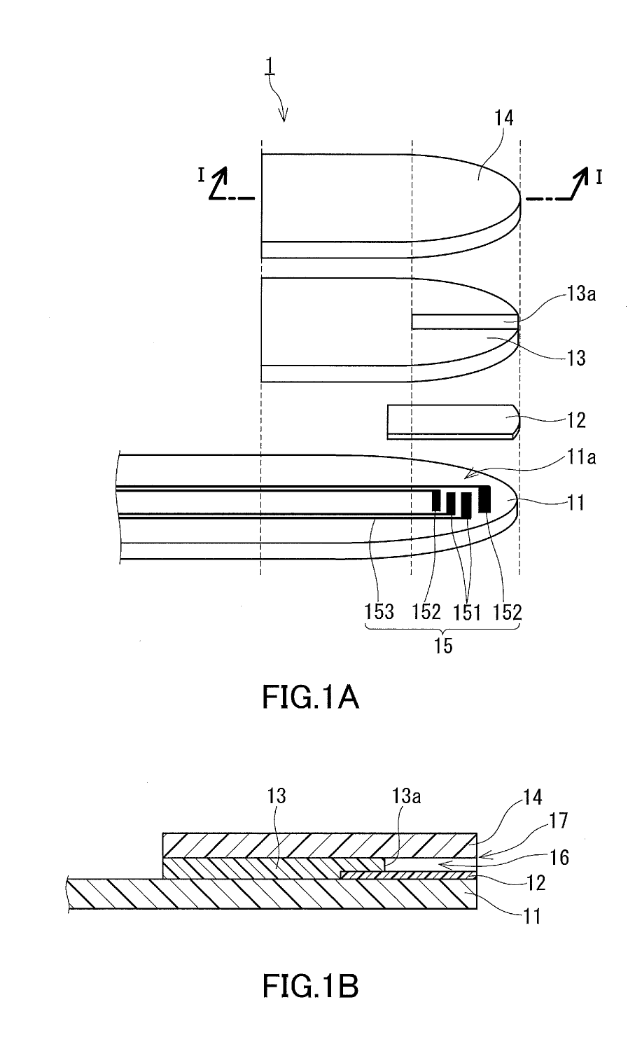

FIG. 1A and FIG. 1B show a configuration example (first configuration example) of the biosensor chip. FIG. 1A is a schematic exploded perspective view of a biosensor chip, and FIG. 1B is a cross-sectional view along the line I-I of FIG. 1A. The biosensor chip 1 shown in FIG. 1A and FIG. 1B includes an electrode substrate 11, a hydrophilic filter 12, a spacer layer 13, and a cover film 14. A first principal surface 11a of the electrode substrate 11 is provided with an electrode pattern 15 including a pair of electrodes (a first electrode 151 and a second electrode 152) and predefined wiring lines 153. The hydrophilic filter 12 is disposed on the first principal surface 11a of the electrode substrate 11 to cover the electrodes 151 and 152. The portions of the electrodes 151 and 152 which are covered by the hydrophilic filter 12 include at least portions positionally corresponding to a slit 13a which is provided in the spacer layer 13 and which is described below; namely, it is sufficient that the hydrophilic filter 12 cover those portions of the electrodes 151 and 152 which are not covered by the spacer layer 13 and which can come into contact with a blood sample. In the first configuration example, the hydrophilic filter 12 extends over the whole of a sample channel 16 described below, has approximately the same shape as the below-described slit 13a of the spacer layer 13, and has a larger size (a slightly larger size in the first configuration example) than the slit 13a. The spacer layer 13 is disposed on the first principal surface 11a of the electrode substrate 11 on which the hydrophilic filter 12 is disposed. The spacer layer 13 is a spacer layer for forming the sample channel 16 and has the slit 13a provided in a region positionally corresponding at least to the electrodes 151 and 152. The spacer layer 13 also serves as a bonding member to join the electrode substrate 11 and the cover film 14 together. The spacer layer 13 is disposed in such a manner that the periphery of the slit 13a is located inwardly of the outer periphery of the hydrophilic filter 12, and the hydrophilic filter 12 is bonded to the electrode substrate 11 by the spacer layer 13. The cover film 14 is disposed on the spacer layer 13 and is opposed to the first principal surface 11a of the electrode substrate 11. A zone defined by the electrode substrate 11, the slit 13a of the spacer layer 13, and the cover film 14 serves as the sample channel 16. The sample channel 16 has an opening at a peripheral surface of a laminate of the electrode substrate 11, the spacer layer 13, and the cover film 14, and this opening is a sample inlet orifice 17 (see FIG. 1(B)). The sample channel 16 also has an air hole (not shown) formed at a position on the opposite side from the sample inlet orifice 17. The blood sample is introduced from the sample inlet orifice 17 deep into the sample channel 16 (to the end opposite from the sample inlet orifice 17) by capillary action and reaches the electrodes 151 and 152 through the hydrophilic filter 12.

Hereinafter, the components of the biosensor chip 1 will be individually described in more detail.

(Electrode Substrate 11)

The electrode substrate 11 can be fabricated by preparing a support substrate having at least one principal surface with insulating properties and by using a conductive material to print on the support substrate the electrode pattern 15 including the first electrode 151, the second electrode 152, and the predefined wiring lines 153. The support substrate used can be a known substrate, such as a resin substrate, which is commonly used as a support substrate in an electrode substrate of a biosensor chip. The support substrate may be multi-layered. In this case, only the outermost layer forming the at least one principal surface needs to be made of a material having insulating properties.

One of the first electrode 151 and second electrode 152 paired with each other serves as a working electrode, while the other serves as a counter electrode. A wiring line connected to the first electrode 151 and a wiring line connected to the second electrode 152 respectively extend to terminals (not shown). The material and method for forming the electrode pattern 15 are not particularly limited, and the electrode pattern 15 can be formed by a known method using a known material which is commonly used in an electrode or the like of a biosensor chip. The electrodes, wiring lines, and terminals need not be made of the same material, and may be formed using different materials. The patterns of the electrodes and wiring lines and the number of the electrodes are not limited to those shown in FIG. 1, and can be appropriately selected depending on, for example, the measurement scheme of the biosensor device. For example, in a variant of the electrode pattern 15, the wiring lines 153 may turn toward the side edges of the electrode substrate 11 instead of extending toward an end of the electrode substrate 11 (variant 1 of the first configuration example). In the variant 1, the direction in which the slit 13a of the spacer layer 13 extends is varied according to the positions of the electrodes 151 and 152. Thus, in the variant 1, the direction in which the sample channel 16 extends is also varied, and the position where the hydrophilic filter 12 is disposed is appropriately varied according to the positions of the electrodes 151 and 152 and the direction in which the slit 13a extends.

On the surface of at least one of the electrodes 151 and 152 that serves as a working electrode there may be a reaction layer (not shown) which is formed, for example, by applying a reagent containing an enzyme and an electron carrier to the surface of the electrode. The actions of the enzyme and electron carrier in the biosensor chip will be briefly described. The following description is given of an example where the component to be measured in the blood sample is blood sugar (glucose). When the blood sample reaches an electrode surface to which the reagent containing the enzyme and electron carrier has been applied, the enzyme oxidizes glucose in the blood, and the enzyme itself is reduced. The enzyme in a reduced state undergoes oxidation-reduction reaction with the electron carrier (oxidized state) and thereby brings the electron carrier into a reduced state. The electron carrier in a reduced state reaches an electrode surface on which a potential is imposed, and the electron carrier undergoes oxidation reaction at the electrode surface, generating a current flowing between the electrodes. The flowing current depends on the glucose concentration in the blood. The glucose concentration in the blood (blood-glucose level) is thus indirectly measured by the current value.

Examples of the enzyme used in the glucose concentration measurement include known enzymes, such as glucose oxidase, glucose dehydrogenase, and glucose dehydrogenase, which are commonly used in biosensors for glucose concentration measurement. Examples of the electron carrier used in the glucose concentration measurement include known electron carriers, such as ferrocene, ferrocene derivatives, quinone, quinone derivatives, conductive organic salts, and hexaammineruthenium(III) chloride, which are commonly used in biosensors for glucose concentration measurement. When a component other than glucose, such as cholesterol, is to be measured, a known enzyme and electron carrier appropriate for the component may be used.

When the enzyme and the electron carrier are contained in the hydrophilic filter 12, the formation of the reaction layer on the surface of the electrode 151 or 152 can be omitted.

(Hydrophilic Filter 12)

The thickness of the hydrophilic filter 12 is preferably 50 μm or less. Controlling the thickness of the hydrophilic filter 12 to 50 μm or less allows the hydrophilic filter 12 to be placed within the sample channel 16 without significantly increasing the size of the sample channel 16 as compared to sample channels of known biosensor chips. Additionally, controlling the thickness of the hydrophilic filter 12 to 50 μm or less prevents the volume of the hydrophilic filter 12 from accounting for too high a proportion in the sample channel 16 and thereby prevents the hydrophilic filter 12 from obstructing the introduction of the blood sample into the sample channel 16. Furthermore, such a thin filter is capable of efficient filtration without pressurization. The use of a hydrophilic filter with a thickness of 50 μm or less can therefore ensure a measurement speed comparable to that of conventional biosensor chips. The lower limit of the thickness of the hydrophilic filter 12 is not particularly defined. The thickness of the hydrophilic filter 12 is preferably 5 μm or more to make the thickness uniform and thus prevent performance variation within the filter.

A porous membrane can be used as the hydrophilic filter 12. For example, the pore diameter of the porous membrane is preferably 5 μm or less, more preferably less than 1 μm, and particularly preferably less than 0.5 μm. The use of a porous membrane having a pore diameter of 5 μm or less as the hydrophilic filter 12 ensures that the hydrophilic filter 12 reliably captures red blood cells in the blood sample. When a porous membrane having a pore diameter of less than 1 μm is used as the hydrophilic filter 12, red blood cells in the blood sample can be captured more reliably. When a porous membrane having a pore diameter of less than 0.5 μm is used as the hydrophilic filter 12, red blood cells in the blood sample can be captured even more reliably. The lower limit of the pore diameter is not particularly defined. In view of the rate of blood permeation, the pore diameter of the porous membrane is preferably 0.05 μm or more.

The material of the hydrophilic filter 12 is not particularly limited, and examples of usable materials include the following resin materials: polyolefin resins such as polyethylene and polypropylene; acrylic or methacrylic resins such as polymethylmethacrylate (PMMA) and polyacrylonitrile (PAN); polyester resins such as polyethylene terephthalate (PET); epoxy resins; polysulfone; polyethersulfone; modified cellulose such as cellulose acetate; cellulose; polyvinylidene fluoride (PVDF); and polytetrafluoroethylene (PTFE). When a porous membrane made of a non-hydrophilic resin material is used, the surface of the porous membrane is subjected to hydrophilization. Exemplary techniques for hydrophilization include: application of a surfactant to the surface of the porous membrane; plasma treatment of the surface of the porous membrane; and coating of the surface of the porous membrane with a hydrophilic material (sizing treatment). The surfactant used for hydrophilization is not particularly limited, and may be appropriately selected from surfactants commonly used in the filed of biotechnology. Examples of the surfactant used in hydrophilization for obtaining the hydrophilic filter 12 include “Triton X-100”, “Triton X-114”, “Tween 20”, “Tween 60”, and “Tween 80” which are non-ionic surfactants. When a porous membrane made of a hydrophilic material is used, hydrophilization is not necessary but may be carried out to increase the hydrophilicity of the membrane.

The hydrophilic filter 12 may contain an enzyme and an electron carrier. The enzyme and the electron carrier are as described above. Incorporation of the enzyme and the electron carrier into the hydrophilic filter 12 eliminates the need to form a reaction layer on the surface of the electrode 151 or 152. This configuration enables the reaction to occur simultaneously with passage of the blood sample through the hydrophilic filter 12 and to uniformly proceed, and thereby yields a higher measurement speed and measurement accuracy than a configuration in which the measurement is based on the reaction that occurs after the blood sample reaches a reaction layer on the surface of the electrode 151 or 152.

As shown in FIG. 1, the hydrophilic filter 12 of the first configuration example extends over the whole sample channel 16, has approximately the same shape as the slit 13a provided in the spacer layer 13, and has a slightly larger size than the slit 13a. The hydrophilic filter 12 is not limited to this form, since it is sufficient that the hydrophilic filter 12 cover at least the electrodes 151 and 152.

The hydrophilic filter 12 shown in FIG. 1 is placed in such a manner that an end of the hydrophilic filter 12 approximately coincides with the tips of the electrode substrate 11, the spacer layer 13, and the cover film 14. Alternatively, the end of the hydrophilic filter 12 may be located outwardly of the tips of the electrode substrate 11, the spacer layer 13, and the cover film 14 (variant 2 of the first configuration example). According to this variant 2, the end of the hydrophilic filter 12 which protrudes from the tip of the chip serves as a blood sample inlet portion, enabling smoother introduction of the blood sample into the sample channel 16.

To allow the hydrophilic filter 12 to cover a wider region including the portion of the electrode substrate 11 where the electrodes 151 and 152 are provided, the hydrophilic filter 12 may, for example, be formed to have the same shape as the tip portion of the electrode substrate 11 and be disposed on the electrode substrate 11 in such a manner that the tip of the electrode substrate 11 and the end of the filter 12 are aligned with each other (variant 3 of the first configuration example). In this case, the hydrophilic filter 12 and the electrode substrate 11 may be bonded with an adhesive by exploiting a region having no electrode pattern 15 in the electrode substrate 11. According to this variant 3, red blood cells can be more effectively removed from the blood sample so that the volume of red blood cells contained in the blood sample reaching the electrodes 151 and 152 can be reduced.

The hydrophilic filter 12 may be secured to the electrode substrate 11, for example, by the steps of: applying a reagent to the surface of the electrode 151 or 152 to form a reaction layer; disposing the hydrophilic filter 12 on the layer of the applied reagent; and then drying the layer of the reagent. In this case, the spacer layer 13 does not need to bond the hydrophilic filter 12 to the electrode substrate 11. Thus, in this case, the shape and size of the hydrophilic filter 12 can be the same as those of the slit 13a of the spacer layer 13, or, for example, the hydrophilic filter 12 can be smaller than the slit 13a so as to extend only over the region positionally corresponding to the electrodes (variant 4 of the first configuration example).

(Spacer Layer 13)

The spacer layer 13 forms the sample channel 16 by the slit 13a. The cross-section of the sample channel 16 is defined depending on the width of the slit 13a and the thickness of the spacer layer 13. The width of the slit 13a can be, for example, 0.2 to 5 mm. The thickness of the spacer layer 13 can be, for example, 0.1 to 1 mm.

The spacer layer 13 bonds the electrode substrate 11, the hydrophilic filter 12, and the cover film 14 to one another and joins them together. Thus, a sheet-shaped bonding member such as a double-coated adhesive tape which includes a sheet substrate having adhesive layers on its both surfaces is suitably used as the spacer layer 13. When such a bonding member is used, the sheet substrate is preferably hydrophilic. The sheet substrate is exposed at the peripheral surface of the slit 13a and faces the sample channel 16; thus, the use of a hydrophilic sheet substrate makes easier the introduction of the blood sample into the sample channel 16.

In the present embodiment, one end of the slit 13a extends to the tip of the spacer layer 13, and the slit 13a opens at the peripheral surface of the spacer layer 13. The slit 13a is not limited to this form, and the one end of the slit 13a need not extend to the tip of the spacer layer 13; namely, the slit 13a need not open at the peripheral surface of the spacer layer 13.

(Cover Film 14)

As the cover film 14 there can be used, for example, a known film such as a polyethylene terephthalate (PET) film which is commonly used as a cover film in a biosensor. As described above, the auxiliary function for introducing the blood sample into the sample channel 16 by capillary action can be performed by the hydrophilic filter 12. Thus, a film not subjected to hydrophilization can also be used as the cover film 14. A groove (not shown) may be provided in the tip portion of the cover film 14 to facilitate the introduction of the blood sample into the sample channel 16 (variant 5 of the first configuration example).

[Second Configuration Example]

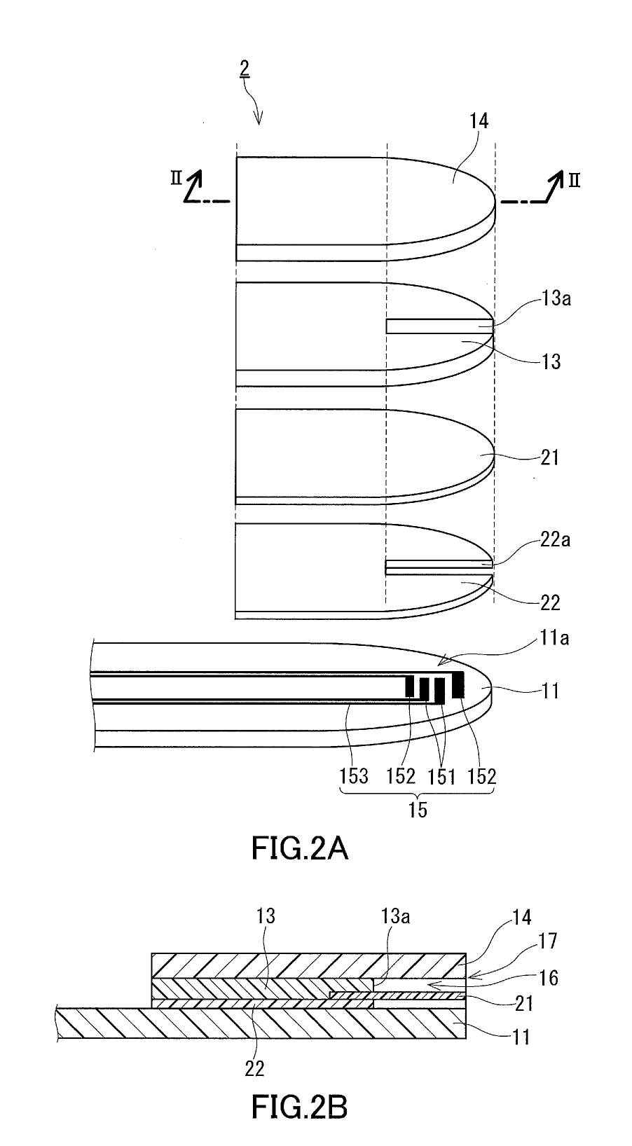

Next, another configuration example (second configuration example) of the biosensor chip according to the present embodiment will be described with reference to FIG. 2A and FIG. 2B. FIG. 2A is a schematic exploded perspective view of a biosensor chip, and FIG. 2B is a cross-sectional view along the line II-II of FIG. 2A. Components identical to those of the biosensor chip 1 of the first configuration example are denoted by the same reference numerals and will not be described again.

The biosensor chip 2 of the second configuration example which is shown in FIG. 2A and FIG. 2B differs from the biosensor chip 1 of FIG. 1 in that the biosensor chip 2 includes a hydrophilic filter 21 having a different shape from the hydrophilic filter 12 and in that the biosensor chip 2 further includes a bonding member 21 disposed between the hydrophilic filter 21 and the electrode substrate 11 to bond the hydrophilic filter 21 to the electrode substrate 11. The description of the biosensor chip 2 is therefore directed only to the hydrophilic filter 21 and the bonding member 22.

(Hydrophilic Filter 21)

The hydrophilic filter 21 has approximately the same outline as the spacer layer 13 and cover film 14. That is, the hydrophilic filter 21 covers a wider region including the portion of the electrode substrate 11 where the electrodes 151 and 152 are provided. The hydrophilic filter 21 is identical to the hydrophilic filter 12 except for the shape, and will therefore not be described further.

(Bonding Member 22)

The bonding member 22 has a slit 22a of approximately the same shape as the slit 13a of the spacer layer 13 in a region positionally corresponding to the slit 13a, namely in a region that overlaps the slit 13a when a laminate of the spacer layer 13 and the bonding member 22 is viewed in the lamination direction. The purpose of the slit 22a is to prevent obstruction of the channel through which the blood sample reaches the electrodes 151 and 152. The space within the slit 22a (opening portion defined by the slit 22a) serves as a through hole forming a part of the sample channel. The hydrophilic filter 21 can thus be firmly secured to the electrode substrate 11 without obstructing the channel through which the blood sample reaches the surfaces of the electrodes 151 and 152. A sheet-shaped bonding member such as a double-coated adhesive tape which includes a sheet substrate having adhesive layers on its both surfaces is suitably used as the bonding member 22. The slit 22a need not have approximately the same shape as the slit 13a, and may have any shape as long as the slit 22a is formed so as to prevent obstruction of the flow of the blood sample toward the electrodes 151 and 152. The bonding member 22 is not limited to the shape shown in FIG. 2. For example, the bonding member 22 may be composed of separate segments so that the bonding member 22 does not obstruct the sample channel 16 (variant 1 of the second configuration example).

[Third Configuration Example]

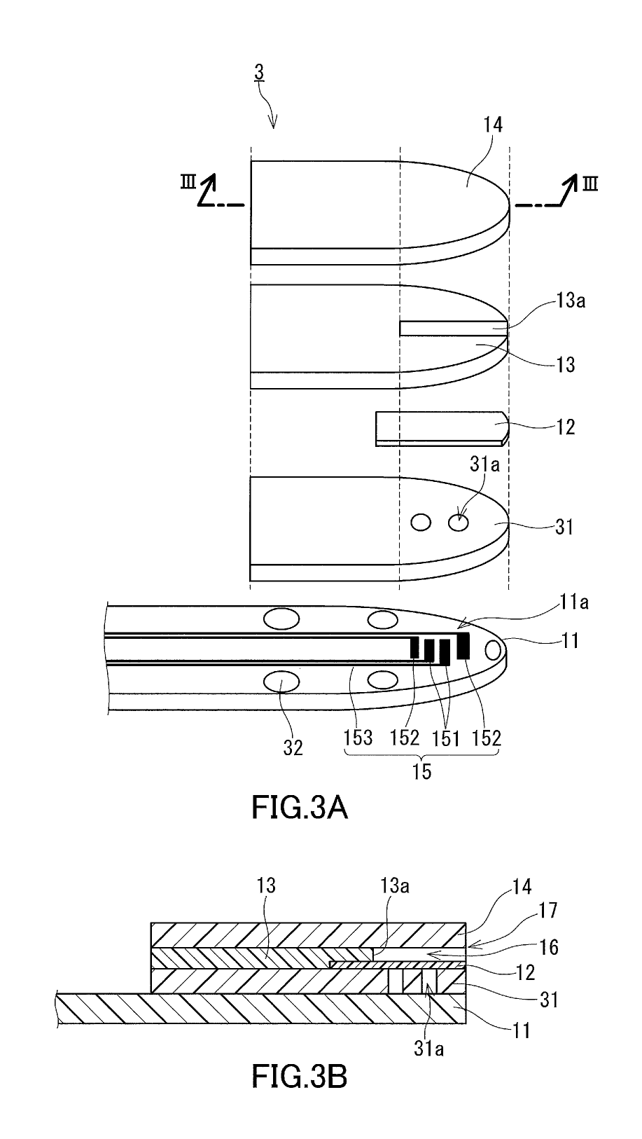

Next, another configuration example (third configuration example) of the biosensor chip according to the present embodiment will be described with reference to FIG. 3A and FIG. 3B. FIG. 3A is a schematic exploded perspective view of a biosensor chip, and FIG. 3B is a cross-sectional view along the line III-III of FIG. 3A. Components identical to those of the biosensor chip 1 of the first configuration example are denoted by the same reference numerals and will not be described again.

The biosensor chip 3 of the third configuration example which is shown in FIG. 3A and FIG. 3B differs from the biosensor chip 1 of FIG. 1 in that the biosensor chip 3 further includes an electrode substrate cover film 31 disposed between the hydrophilic filter 12 and the electrode substrate 11 and covering the tip portion of the electrode substrate 11 and in that the electrode substrate cover film 31 is bonded to the electrode substrate 11 by an adhesive 32. The description of the biosensor chip 2 is therefore directed only to the electrode substrate cover film 31.

(Electrode Substrate Cover Film 31)

The outline of the electrode substrate cover film 31 is approximately the same as the outline of the tip portion of the electrode substrate 11, and the electrode substrate cover film 31 covers the tip portion of the electrode substrate 11. The electrode substrate cover film 31 is provided with an opening 31a in a region overlapping the electrodes 151 and 152 (a region that overlaps at least some portions of the electrodes 151 and 152 when a laminate of the electrode substrate 11 and the electrode substrate cover film 31 is viewed in the lamination direction), and this opening 31a prevents the electrode substrate cover film 31 from obstructing the channel through which the blood sample reaches the surfaces of the electrodes 151 and 152. As the electrode substrate cover film 31 there can be used, for example, a film such as a PET film which can be used as the cover film 14. The thickness of the electrode substrate cover film 31 is not particularly limited, and may be, for example, 50 to 300 μm.

[Fourth Configuration Example]

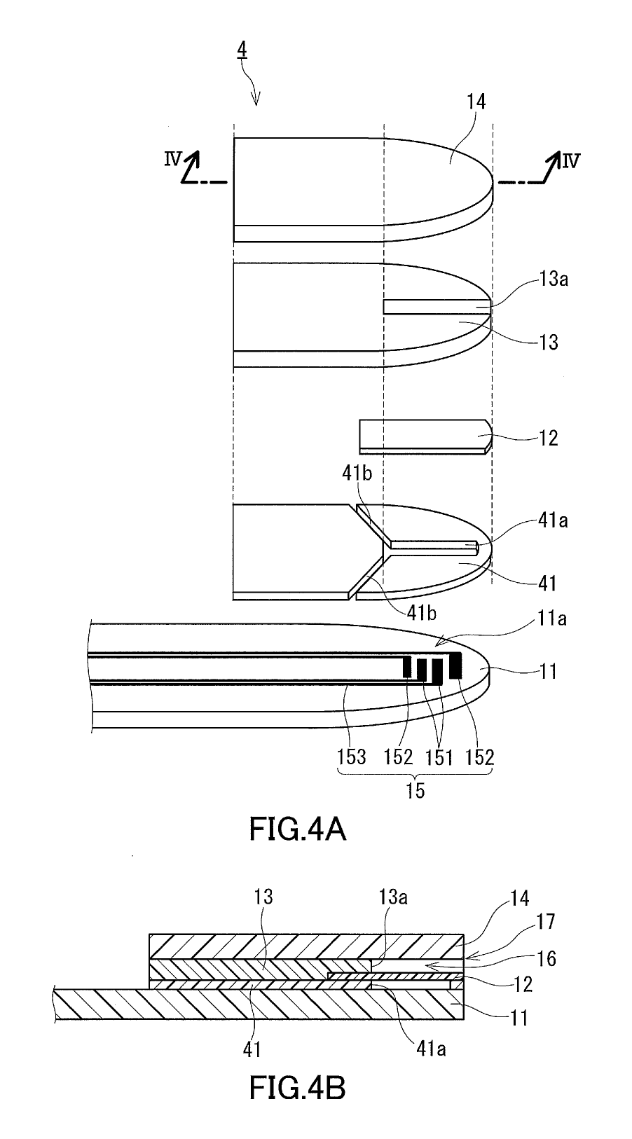

Next, another configuration example (fourth configuration example) of the biosensor chip according to the present embodiment will be described with reference to FIG. 4A and FIG. 4B. FIG. 4A is a schematic exploded perspective view of a biosensor chip, and FIG. 4B is a cross-sectional view along the line IV-IV of FIG. 4A. Components identical to those of the biosensor chip 1 of the first configuration example are denoted by the same reference numerals and will not be described again.

The biosensor chip 4 of the fourth configuration example which is shown in FIG. 4A and FIG. 4B differs from the biosensor chip 1 of FIG. 1 in that the biosensor chip 4 further includes a bonding member 41 disposed between the hydrophilic filter 12 and the electrode substrate 11 to bond the hydrophilic filter 12 to the electrode substrate 11. The description of the biosensor chip 4 is therefore directed only to the bonding member 41.

(Bonding Member 41)

The bonding member 41 has a slit 41a of approximately the same shape as the slit 13a of the spacer layer 13 in a region positionally corresponding to the slit 13a, namely in a region that overlaps the slit 13a when a laminate of the spacer layer 13 and the bonding member 41 is viewed in the lamination direction. The purpose of the slit 41a is to prevent obstruction of the channel through which the blood sample reaches the electrodes 151 and 152. The space within the slit 41a (opening portion defined by the slit 41a) serves as a through hole forming a part of the sample channel. The slit 41a provided in the bonding member 41 differs from the slit 13a of the spacer layer 13 in that an end of the slit 41a does not extend to the tip of the bonding member 41 but is closed without opening at the peripheral surface of the bonding member 41. The slit 41a provided in the bonding member 41 allows the hydrophilic filter 12 to be firmly secured to the electrode substrate 11 without obstructing the channel through which the blood sample reaches the surfaces of the electrodes 151 and 152. The bonding member 41 is further provided with a vent hole 41b communicating with the space within the slit 41a. The provision of such a vent hole 41b makes it possible, when the sample permeates the hydrophilic filter 12, to discharge air from the space within the slit 41a to the outside of the chip 4 through the vent hole 41b, despite the fact that the bonding member 41 used has a configuration in which an end of the slit 41a (the end nearer the tip of the chip 4) is closed instead of extending to the tip of the bonding member 41. This prevents the permeation of the sample through the hydrophilic filter 12 from being slowed. A sheet-shaped bonding member such as a double-coated adhesive tape which includes a sheet substrate having adhesive layers on its both surfaces is suitably used as the bonding member 41. The slit 41a need not have approximately the same shape as the slit 13a, and may have any shape as long as the slit 41a is formed so as to prevent obstruction of the flow of the blood sample toward the electrodes 151 and 152. The bonding member 41 is not limited to the shape shown in FIG. 4. For example, the bonding member 41 may be composed of separate segments so that the bonding member 41 does not obstruct the sample channel 16 (variant 1 of the fourth configuration example). The shape of the vent hole 41b of the bonding member 41 is not particularly limited, as long as the vent hole 41b allows gas vent without causing leakage of crystals. Thus, the bonding member 41 may be provided with two or more vent holes 41b as shown in FIG. 4A or may be provided with one vent hole 41b.

[Fifth Configuration Example]

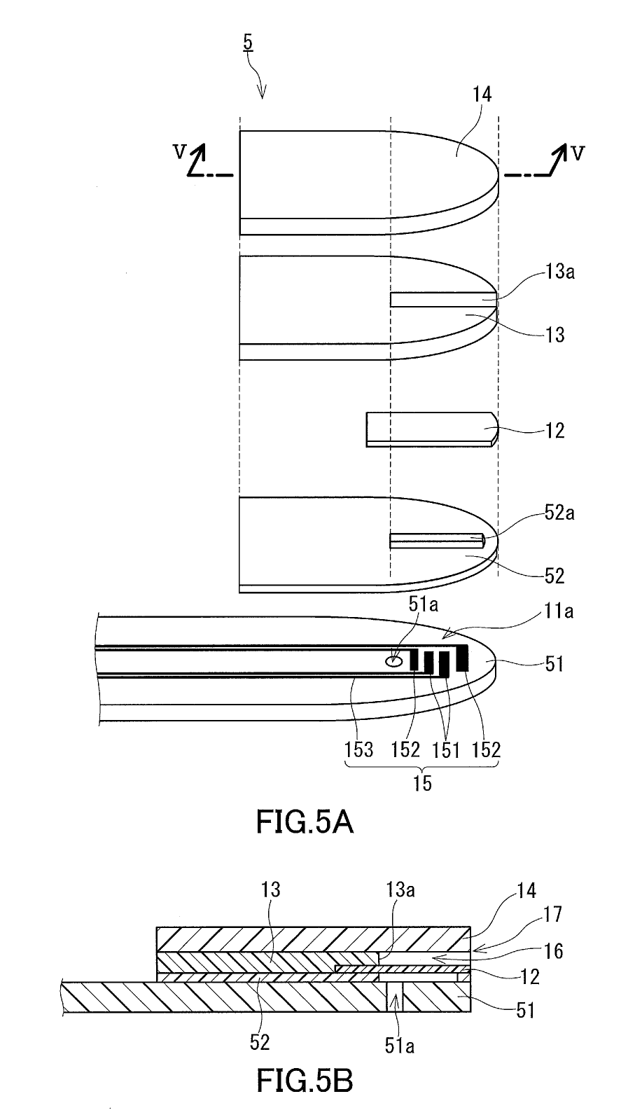

Next, another configuration example (fifth configuration example) of the biosensor chip according to the present embodiment will be described with reference to FIG. 5A and FIG. 5B. FIG. 5A is a schematic exploded perspective view of a biosensor chip, and FIG. 5B is a cross-sectional view along the line V-V of FIG. 5A. Components identical to those of the biosensor chip 1 of the first configuration example are denoted by the same reference numerals and will not be described again.

The biosensor chip 5 of the fifth configuration example which is shown in FIG. 5A and FIG. 5B differs from the biosensor chip 1 of FIG. 1 in that the biosensor chip 5 includes an electrode substrate 51 having a different shape from the electrode substrate 11 and in that the biosensor chip 5 further includes a bonding member 52 disposed between the hydrophilic filter 12 and the electrode substrate 51 to bond the hydrophilic filter 12 to the electrode substrate 51. The description of the biosensor chip 5 is therefore directed only to the electrode substrate 51 and the bonding member 52. For convenience of explanation, the bonding member 52 will be described first, followed by the electrode substrate 51.

(Bonding Member 52)

The bonding member 52 has a slit 52a of approximately the same shape as the slit 13a of the spacer layer 13 in a region positionally corresponding to the slit 13a, namely in a region that overlaps the slit 13a when a laminate of the spacer layer 13 and the bonding member 52 is viewed in the lamination direction. The purpose of the slit 52a is to prevent obstruction of the channel through which the blood sample reaches the electrodes 151 and 152. The space within the slit 52a (opening portion defined by the slit 52a) serves as a through hole forming a part of the sample channel. The slit 52a provided in the bonding member 52 differs from the slit 13a of the spacer layer 13 in that an end of the slit 52a does not extend to the tip of the bonding member 52 but is closed without opening at the peripheral surface of the bonding member 52. The slit 52a provided in the bonding member 52 allows the hydrophilic filter 12 to be firmly secured to the electrode substrate 11 without obstructing the channel through which the blood sample reaches the surfaces of the electrodes 151 and 152. A sheet-shaped bonding member such as a double-coated adhesive tape which includes a sheet substrate having adhesive layers on its both surfaces is suitably used as the bonding member 52. The slit 52a need not have approximately the same shape as the slit 13a, and may have any shape as long as the slit 52a is formed so as to prevent obstruction of the flow of the blood sample toward the electrodes 151 and 152. The bonding member 52 is not limited to the shape shown in FIG. 5. For example, the bonding member 52 may be composed of separate segments so that the bonding member 52 does not obstruct the sample channel 16 (variant 1 of the fifth configuration example).

(Electrode Substrate 51)

The electrode substrate 51 is provided with a vent hole 51a extending through the thickness of the electrode substrate 51. The electrode substrate 51 has the same configuration as the electrode substrate 11 except for having the vent hole 51a, and only the vent hole 51a will therefore be described now. The vent hole 51a is positioned so that its internal space can communicate with the space within the slit 52a provided in the bonding member 52. The provision of such a vent hole 51a in the electrode substrate 51 makes it possible, when the sample permeates the hydrophilic filter 12, to discharge air from the space within the slit 52a to the outside of the chip 5 through the vent hole 51b, despite the fact that the bonding member 52 used has a configuration in which an end of the slit 52a (the end nearer the tip of the chip 5) is closed instead of extending to the tip of the bonding member 52. This prevents the permeation of the sample through the hydrophilic filter 12 from being slowed. The shape of the vent hole 51a of the electrode substrate 51 is not particularly limited, as long as the vent hole 51a allows gas vent without impairing the function of the electrode substrate. Thus, the electrode substrate 51 may be provided with one vent hole 51a as shown in FIG. 5A or may be provided with two or more vent holes 51a.

The foregoing has described various configuration examples of the biosensor chip according to the present embodiment; however, the biosensor chip according to the present invention is not limited to the above configuration examples. For example, a sensing portion that senses a blood sample is provided, instead of the electrodes 151 and 152, as the blood sample sensing means in the electrode substrate 11 or 51. The slit 13a is not limited to straight slits as shown in FIGS. 1A, 2A, 3A, 4A, and 5A and may have any shape that allows introduction of the blood sample by capillary action. For example, the slit 13a may be curved or zig-zagged or may be formed of a combination of a straight segment, a curved segment, and a zig-zag segment.

The biosensor chip according to the present invention is not limited to the present embodiment. The biosensor chip according to the present invention encompasses, for example, biosensor chips A and B as defined below and can be implemented with various modifications falling within the scope of the biosensor chips A and B as defined below.

(Biosensor Chip A)

A biosensor chip including:

a substrate having a first principal surface provided with an electrode;

a cover film opposed to the first principal surface of the substrate; and

a spacer layer disposed between the substrate and the cover film and serving as a bonding member to join the substrate and the cover film together, wherein

the spacer layer is provided with a slit forming: a sample inlet orifice provided at a peripheral surface of a laminate of the substrate, the spacer layer, and the cover film; and a sample channel for delivering a sample to the electrode by capillary action, and

a hydrophilic filter is provided between the slit of the spacer layer and a sample sensing portion of the electrode of the substrate.

(Biosensor Chip B)

A biosensor chip including:

a substrate having a first principal surface provided with a sensing portion that senses a blood sample;

a cover film opposed to the first principal surface of the substrate;

a spacer layer disposed between the substrate and the cover film, the spacer layer having a sample channel into which the blood sample is introduced by capillary action, the spacer layer serving as a bonding member to join the substrate and the cover film together; and

a hydrophilic filter disposed between the spacer layer and the substrate and located at a position through which the blood sample passes to reach the sensing portion.

[Biosensor Device]

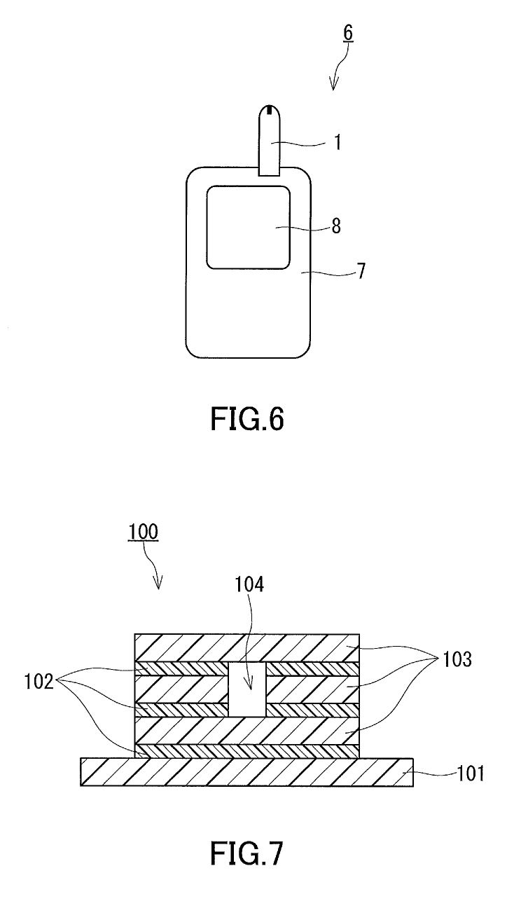

Next, an embodiment of the biosensor device according to the present invention will be described. As shown in FIG. 6, a biosensor device 6 according to the present embodiment includes a device body 7 and the biosensor chip 1 shown in FIG. 1 which is detachably attached to the device body 7. The device body 7 includes: a detection portion (not shown) that detects a substance in a sample on the basis of the value of a current flowing between the pair of electrodes 151 and 152 of the biosensor chip 1; an analysis portion (not shown) that analyzes a detection result obtained by the detection portion; and a display portion 8 that displays as a measurement value an analysis result obtained by the analysis portion. In the biosensor device 6, the biosensor chip 2,3,4, or 5 can be used instead of the biosensor chip 1.

The foregoing has described a configuration example in which the biosensor chip is detachably attached to the device body of the biosensor device, namely in which only the biosensor chip is a disposable part. However, the present invention is not limited to this configuration. For example, the biosensor chip itself may further include: a detection portion that detects a substance in a sample on the basis of the value of a current flowing between the pair of electrodes; an analysis portion that analyzes a detection result obtained by the detection portion; and a display portion that displays as a measurement value an analysis result obtained by the analysis portion. In this case, the biosensor chip itself can serve as a measurement device that requires no device body. When the biosensor chip itself serves as a measurement device, the measurement device can itself be disposable.

### EXAMPLES

Next, the biosensor chip according to the present invention will be specifically described with examples.

[Fabrication of Filter]

(Filter A)

In a 3 L cylindrical plastic container, 100 parts by weight of jER (registered trademark) 828 (bisphenol A-type epoxy resin manufactured by Mitsubishi Chemical Corporation and having an epoxy equivalent of 184 to 194 g/eq.) and 25 parts by weight of TETRAD (registered trademark)-C(glycidylamine-type epoxy resin manufactured by Mitsubishi Gas Chemical Company, Inc. and having an epoxy equivalent of 95 to 110 g/eq.) were dissolved in 211.9 parts by weight of polypropylene glycol (Adeka Polyether P-400 manufactured by ADEKA Corporation) to prepare an epoxy resin/polypropylene glycol solution. After that, 22.3 parts by weight of 1,6-diaminohexane was added to the plastic container to prepare an epoxy resin/amine/polypropylene glycol solution. Next, using a planetary centrifugal mixer (manufactured by Thinky Corporation under the trade name “Awatori Rentaro (registered trademark)”), the solution was vacuum-degassed at about 0.7 kPa while being stirred at a revolution speed of 800 rpm and a rotation/revolution ratio of 3/4 for 10 minutes. This process was repeated twice. This was followed by natural cooling for several days, after which the resulting epoxy resin block was taken out of the plastic container and was sliced continuously at a thickness of 16 μm using a cutting lathe to obtain an epoxy resin sheet. This epoxy resin sheet was washed by immersion in RO water heated to 40° C. and further washed by immersion in RO water at 80° C. The washed epoxy resin sheet was immersed in a 0.5 vol % aqueous solution of polyoxyethylene (10) octylphenyl ether to hydrophilize the epoxy resin sheet, from the surface of which the solution was removed and which was then air-dried. The porous epoxy resin membrane thus obtained was used as a filter A. The obtained filter A had a pore diameter of 0.4 μm.

(Filter B)

A filter B was fabricated in the same manner as the filter A, except for omitting the hydrophilization using the aqueous solution of polyoxyethylene (10) octylphenyl ether.

(Filter C)

A filter C was fabricated in the same manner as the filter A, except for carrying out hydrophilization using, instead of the 0.5 vol % aqueous solution of polyoxyethylene (10) octylphenyl ether, a solution prepared by dissolving 50 mg of glucose oxidase GO-NA (manufactured by Amano Enzyme Inc.) in 10 g of a 0.5 vol % aqueous solution of “Tween 60”.

### Reference Example A

(Fabrication of Test Cell)

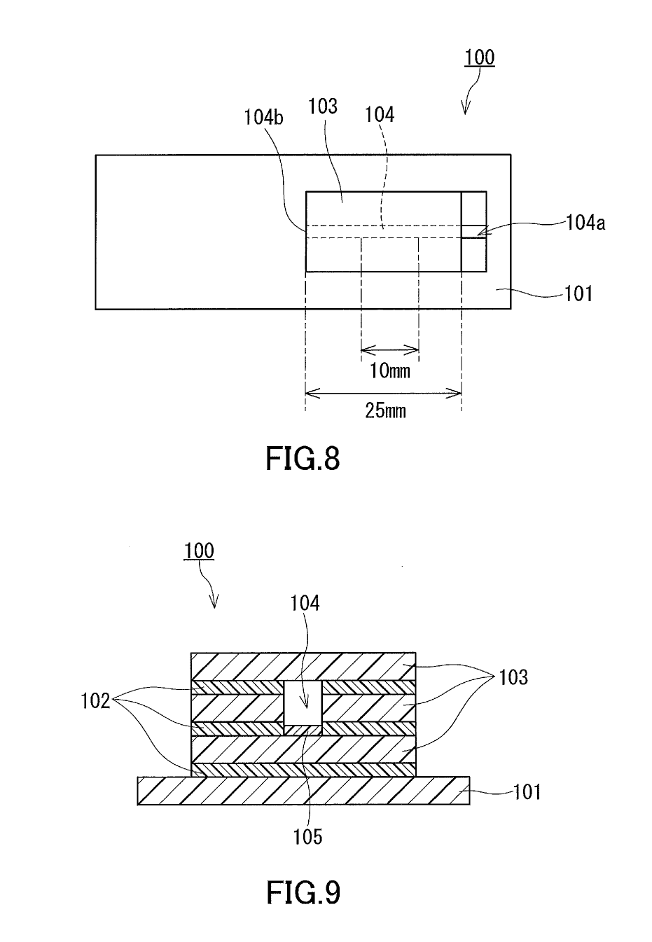

A test cell 100 provided with a channel and having a cross-sectional structure as shown in FIG. 7 was fabricated on a glass slide using a 120-μm-thick double-coated adhesive tape (No. 5015, manufactured by Nitto Denko Corporation) and a polypropylene (PP) film (thickness: 200 μm). In FIG. 7, the numeral 101 denotes the glass slide, the numeral 102 denotes the double-coated adhesive tape, the numeral 103 denotes the PP film, and the numeral 104 denotes the channel. FIG. 8 is a top view of this test cell 100. To allow entry of water into the channel 104, one opening of the channel 104 was used as a water inlet orifice 104a and the other opening was used as an air hole 104b. The channel 104 had a width of 1 mm and a length of 25 mm. A drop of about 20 μL of RO water was applied to the inlet orifice of the channel 104 at room temperature, and the time taken for the RO water to move through a 10-mm-long central region of the channel 104 having an overall length of 25 mm was measured, and the measured time was defined as the penetration time. The contact angle of RO water on the PP film used was 103°, which means that the PP film was sufficiently hydrophobic.

### Reference Example 1

The filter A was cut into a piece of the same shape as the channel 104 of the test cell 100. This piece was placed as a filter 105 inside the channel 104 as shown in FIG. 9, and the penetration time was measured. The penetration time was 0.8 seconds.

### Comparative Reference Example 1

The test cell 100 as shown in FIG. 7 was used by itself to measure the penetration time; namely, measurement of the penetration time was attempted without placing anything in the channel 104. However, RO water failed to penetrate through the channel 104, and the penetration time was not able to be measured.

### Comparative Reference Example 2

The filter B was cut into a piece of the same shape as the channel 104 of the test cell 100. This piece was placed as a filter 105 inside the channel 104 as shown in FIG. 9, and measurement of the penetration time was attempted. However, RO water failed to penetrate through the channel 104, and the penetration time was not able to be measured.

The results for Reference Example 1 and Comparative Reference Examples 1 and 2 confirmed that when a hydrophilic liquid such as RO water is introduced into a channel of a test cell, a hydrophilic filter effectively serves as a member that promotes capillary action.

### Reference Example B

(Fabrication of Test Cell)

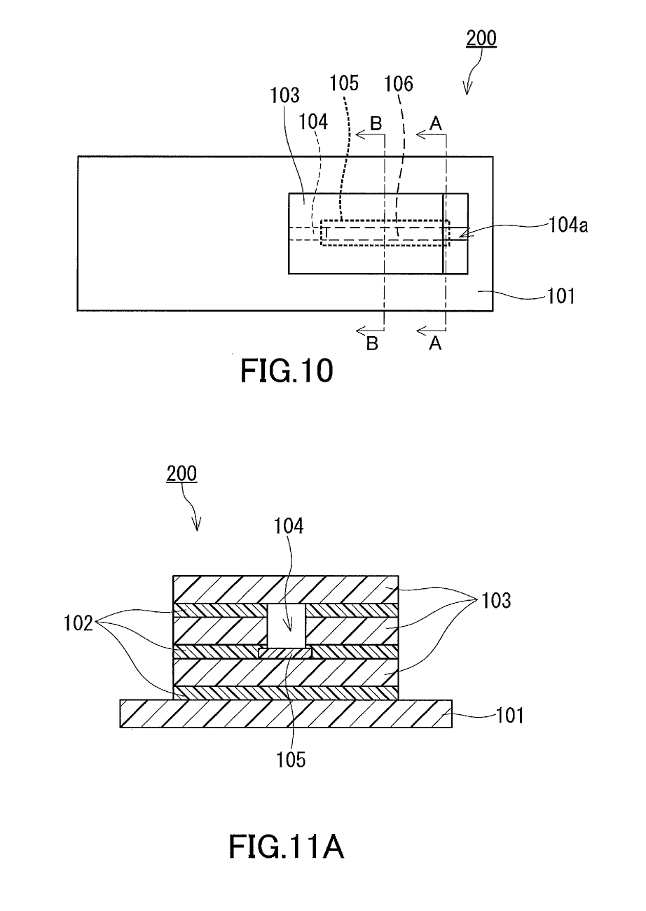

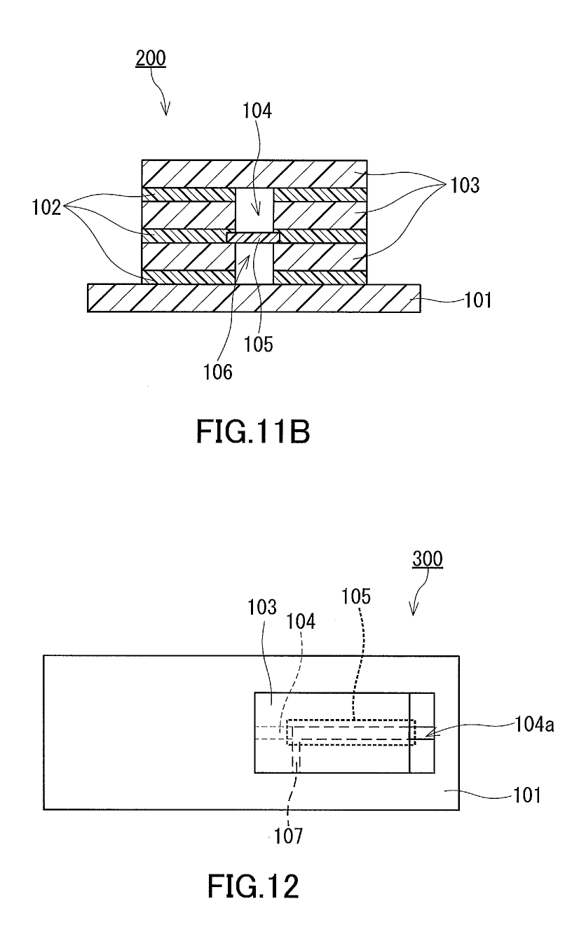

A test cell 200 provided with a channel and having a structure as shown in the top view of FIG. 10 and the cross-sectional views of FIGS. 11A and 11B was fabricated using components identical to those of the test cell 100 of Reference Example A. FIG. 11A is a cross-sectional view along the line A-A of FIG. 10, and FIG. 11B is a cross-sectional view along the line B-B of FIG. 10. FIG. 10 and FIGS. 11A and 11B show a state where the filter 105 is placed in the test cell 200. The test cell 200, unlike the test cell 100, further had a channel 106 having a width of 1 mm and provided below the position where the filter 105 was placed. The test cell 200 had the same structure as the test cell 100, except that the channel 106 was provided. This channel 106 is a zone entered by water permeating the filter 105 when a drop of water is applied to the inlet orifice 104a of the test cell 200. This channel is therefore referred to as “permeate-side channel 106” hereinafter. Furthermore, a test cell 300 was also fabricated by providing the test cell 200 with an air hole 107 having a width of about 0.5 mm and communicating with the internal space of the permeate-side channel 106. FIG. 12 is a top view showing a state where the filter 105 is placed in the test cell 300.

### Reference Example 2

As shown in FIG. 11B, the filter A was disposed to cover the permeate-side channel 106 of the test cell 200, secured to the test cell 200 with a double-coated adhesive tape, and thus used as the filter 105. A drop of about 20 μL of RO water was applied to the inlet orifice 104a of the channel 104 at room temperature to examine the degree of RO water penetration into the permeate-side channel 106. RO water entered the permeate-side channel 106 by permeating the filter A, but failed to fully fill the permeate-side channel 106, in which air bubbles were finally left.

### Reference Example 3

As in Reference Example 2, the filter A was disposed to cover the permeate-side channel 106 of the test cell 300, secured to the test cell 300 with a double-coated adhesive tape, and thus used as the filter 105. A drop of about 20 μL of RO water was applied to the inlet orifice 104a of the channel 104 at room temperature to examine the degree of RO water penetration into the permeate-side channel 106. RO water permeated the filter A and quickly entered the permeate-side channel 106, thereby successfully filling the permeate-side channel 106 without leaving air bubbles.

The results for Reference Examples 2 and 3 confirmed that when a channel is provided on the water permeation side with respect to the hydrophilic filter, it is preferable to provide an air hole, namely a vent hole, to allow efficient entry of a hydrophilic liquid such as RO water into the channel.

### Example 1

A commercially-available biosensor chip for blood-glucose level measurement (manufactured by TaiDoc Technology Corporation) was prepared. This biosensor chip has a sample channel with a width of 1 mm, a length of 5 mm, and a height of 200 μm. A cover film on the top surface of the biosensor chip was removed, a PP film as used in the test cell 100 was attached to the top surface, and an air hole was formed. The filter A was cut into a 1-mm-wide, 5-mm-long piece, which was set within the sample channel so that an end of the filter was aligned with an end of the sample channel. A biosensor chip of Example 1 was thus fabricated. That is, in the biosensor chip of Example 1, the cover film was a hydrophobic film, and a hydrophilic filter was placed within the sample channel. Blood of an adult male was applied to the inlet orifice of the sample channel of the biosensor chip, and the time taken for the blood to pass through the channel length of 5 mm was measured. The blood was drawn into the sample channel and penetrated the 5-mm-long sample channel completely in 0.4 seconds. With the biosensor chip having a hydrophilic filter placed within the sample channel, the filter successfully removed red blood cells from the blood moving toward the electrode. Additionally, the blood smoothly penetrated the sample channel without being obstructed, despite the hydrophobicity of the cover film and the presence of the filter within the sample channel.

### Example 2

A biosensor chip was fabricated in the same manner as in Example 1, except for using the filter C instead of the filter A. Blood of an adult male was applied to the inlet orifice, and the time taken for the blood to pass through the channel length of 5 mm was measured. The blood penetrated the sample channel completely in 0.5 seconds.

### Comparative Example 1

The commercially-available biosensor chip for blood-glucose level measurement which was used in Example 1 was prepared. A cover film on the top surface of the biosensor chip was removed, a PP film as used in the test cell 100 was attached to the top surface, and an air hole was formed. Thus, a biosensor chip of Comparative Example 1 was obtained in which the cover film was a hydrophobic film. Blood of an adult male was applied to the inlet orifice of the sample channel of the biosensor chip. The blood remained adhered in the vicinity of the inlet orifice and failed to penetrate the sample channel.

### INDUSTRIAL APPLICABILITY

The biosensor chip and biosensor device according to the present invention are capable, for example, of measuring the concentration of a component (blood glucose, for example) in a blood sample with improved accuracy and are therefore useful as a chip and device for SMBG.

1. A biosensor chip comprising:
a substrate having a first principal surface provided with an electrode;
a cover film opposed to the first principal surface of the substrate; and
a spacer layer disposed between the substrate and the cover film and serving as a bonding member to join the substrate and the cover film together, wherein
the spacer layer is provided with a slit forming: a sample inlet orifice provided at a peripheral surface of a laminate of the substrate, the spacer layer, and the cover film; and a sample channel for delivering a sample to the electrode by capillary action, and
a hydrophilic filter is provided between the slit of the spacer layer and a sample sensing portion of the electrode of the substrate.

2. A biosensor chip comprising:
a substrate having a first principal surface provided with an electrode;
a cover film opposed to the first principal surface of the substrate;
a spacer layer disposed between the substrate and the cover film, the spacer layer having a slit provided in a region positionally corresponding at least to the electrode, the spacer layer serving as a bonding member to join the substrate and the cover film together; and
a hydrophilic filter disposed between the spacer layer and the substrate and covering at least a portion of the electrode, the portion of the electrode positionally corresponding to the slit, wherein
a zone defined by the cover film, the slit of the spacer layer, and the substrate serves as a sample channel.

3. The biosensor chip according to claim 2, wherein a sample inlet orifice of the sample channel is an opening of the sample channel, the opening being at a peripheral surface of a laminate of the substrate, the spacer layer, and the cover film.

4. A biosensor chip comprising:
a substrate having a first principal surface provided with a sensing portion that senses a blood sample;
a cover film opposed to the first principal surface of the substrate;
a spacer layer disposed between the substrate and the cover film, the spacer layer having a sample channel into which the blood sample is introduced by capillary action, the spacer layer serving as a bonding member to join the substrate and the cover film together; and
a hydrophilic filter disposed between the spacer layer and the substrate and located at a position through which the blood sample passes to reach the sensing portion.

5. The biosensor chip according to claim 1, further comprising a bonding member disposed between the substrate and the hydrophilic filter to bond the hydrophilic filter to the substrate.

6. The biosensor chip according to claim 5, wherein the bonding member has: a through hole forming a part of the sample channel; and a vent hole communicating with an interior of the through hole.

7. The biosensor chip according to claim 5, wherein
the bonding member has a through hole forming a part of the sample channel, and
the substrate has a vent hole communicating with an interior of the through hole of the bonding member.

8. The biosensor chip according to claim 1, wherein the hydrophilic filter has a thickness in the range of 5 μm to 50 μm.

9. The biosensor chip according to claim 1, wherein the hydrophilic filter comprises an enzyme and an electron carrier.

10. The biosensor chip according to claim 1, wherein a reaction layer comprising an enzyme and an electron carrier is provided on a surface of the electrode or of the sensing portion.

11. The biosensor chip according to claim 1, wherein the hydrophilic filter is a porous membrane of at least one selected from polyolefin resin, acrylic resin, methacrylic resin, polyester resin, epoxy resin, polyvinylidene fluoride, polytetrafluoroethylene, polysulfone, polyethersulfone, modified cellulose, and cellulose.

12. The biosensor chip according to claim 1, further comprising:
a detection portion that detects a substance in a sample;
an analysis portion that analyzes a detection result obtained by the detection portion; and
a display portion that displays as a measurement value an analysis result obtained by the analysis portion.

13. A biosensor device comprising:
a device body; and
the biosensor chip according to claim 1, the biosensor chip being detachably attached to the device body, wherein
the device body comprises:
a detection portion that detects a substance in a sample sensed by the biosensor chip;
an analysis portion that analyzes a detection result obtained by the detection portion; and
a display portion that displays as a measurement value an analysis result obtained by the analysis portion.

14. The biosensor chip according to claim 2, further comprising a bonding member disposed between the substrate and the hydrophilic filter to bond the hydrophilic filter to the substrate.

15. The biosensor chip according to claim 14, wherein the bonding member has: a through hole forming a part of the sample channel; and a vent hole communicating with an interior of the through hole.

16. The biosensor chip according to claim 14, wherein
the bonding member has a through hole forming a part of the sample channel, and
the substrate has a vent hole communicating with an interior of the through hole of the bonding member.

17. The biosensor chip according to claim 2, wherein the hydrophilic filter has a thickness in the range of 5 μm to 50 μm.

18. The biosensor chip according to claim 2, wherein the hydrophilic filter comprises an enzyme and an electron carrier.

19. The biosensor chip according to claim 2, wherein a reaction layer comprising an enzyme and an electron carrier is provided on a surface of the electrode or of the sensing portion.

20. The biosensor chip according to claim 2, wherein the hydrophilic filter is a porous membrane of at least one selected from polyolefin resin, acrylic resin, methacrylic resin, polyester resin, epoxy resin, polyvinylidene fluoride, polytetrafluoroethylene, polysulfone, polyethersulfone, modified cellulose, and cellulose.

21. The biosensor chip according to claim 2, further comprising:
a detection portion that detects a substance in a sample;
an analysis portion that analyzes a detection result obtained by the detection portion; and
a display portion that displays as a measurement value an analysis result obtained by the analysis portion.

22. A biosensor device comprising:
a device body; and
the biosensor chip according to claim 2, the biosensor chip being detachably attached to the device body, wherein
the device body comprises:
a detection portion that detects a substance in a sample sensed by the biosensor chip;
an analysis portion that analyzes a detection result obtained by the detection portion; and
a display portion that displays as a measurement value an analysis result obtained by the analysis portion.

23. The biosensor chip according to claim 4, further comprising a bonding member disposed between the substrate and the hydrophilic filter to bond the hydrophilic filter to the substrate.

24. The biosensor chip according to claim 23, wherein the bonding member has: a through hole forming a part of the sample channel; and a vent hole communicating with an interior of the through hole.

25. The biosensor chip according to claim 23, wherein
the bonding member has a through hole forming a part of the sample channel, and
the substrate has a vent hole communicating with an interior of the through hole of the bonding member.

26. The biosensor chip according to claim 4, wherein the hydrophilic filter has a thickness in the range of 5 μm to 50 μm.

27. The biosensor chip according to claim 4, wherein the hydrophilic filter comprises an enzyme and an electron carrier.

28. The biosensor chip according to claim 4, wherein a reaction layer comprising an enzyme and an electron carrier is provided on a surface of the electrode or of the sensing portion.

29. The biosensor chip according to claim 4, wherein the hydrophilic filter is a porous membrane of at least one selected from polyolefin resin, acrylic resin, methacrylic resin, polyester resin, epoxy resin, polyvinylidene fluoride, polytetrafluoroethylene, polysulfone, polyethersulfone, modified cellulose, and cellulose.

30. The biosensor chip according to claim 4, further comprising:
a detection portion that detects a substance in a sample;
an analysis portion that analyzes a detection result obtained by the detection portion; and
a display portion that displays as a measurement value an analysis result obtained by the analysis portion.

31. A biosensor device comprising:
a device body; and
the biosensor chip according to claim 4, the biosensor chip being detachably attached to the device body, wherein
the device body comprises:
a detection portion that detects a substance in a sample sensed by the biosensor chip;
an analysis portion that analyzes a detection result obtained by the detection portion; and
a display portion that displays as a measurement value an analysis result obtained by the analysis portion.
